(19) United States
(12) Patent Application Publication
(10) Pub. No.: US 2026/0294451 A1
(43) Pub. Date: Oct. 1, 2026

(54) METHOD AND APPARATUS FOR PENETRATING CHRONICAL TOTAL OCCLUSIONS (CTOS) IN ARTERIES

(71) Applicant: Physical Sciences, Inc., Andover, MA (US)

(72) Inventors: Nicusor Iftimia, Salem, NH (US); John Grimble, Malden, MA (US)

(21) Appl. No.: 19/559,477

(22) Filed: Mar. 6, 2026

Related U.S. Application Data
(60) Provisional application No. 63/777,283, filed on Mar. 25, 2025.

(51) Int. Cl. A61B 17/22

(52) U.S. Cl. CPC A61B 17/22012; A61B 2017/22068; A61B 2017/22094

(57) ABSTRACT

An occlusion penetrating system and method including a probe with an impact member, an actuator for vibrating the impact member, and a sensor configured to measure the impact force of the impact member. A catheter delivers the impact member within a tube (e.g., vasculature), a power supply is connected to the actuator for energizing the actuator, and a processing subsystem is responsive to the measured impact force of the impact member and is configured to ascertain whether the impact member is impacting the tube or an occlusion (e.g., plaque) in the tube and to automatically deenergize the actuator when the measured impact force indicates the impact member is impacting the tube.

### RELATED APPLICATIONS

This application claims benefit of and priority to U.S. Provisional Application Ser. No. 63/777,283 filed Mar. 25, 2025, under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract No. 1R43HL156359-01A1 awarded by the National Institute of Health. The Government has certain rights in the subject invention.

### FIELD OF THE INVENTION

This subject invention relates to a novel approach and apparatus enabling treatment of chronic total occlusions (CTOs) in arteries.

### BACKGROUND OF THE INVENTION

Peripheral artery disease (also called PAD) is a common circulatory problem in which narrowed arteries reduce blood flow to limbs. In patients with peripheral arterial occlusive disease (PAOD), perfusion is severely impaired due to narrowing (stenosis) or complete blockage (occlusion) of the arterial lumen. More than 200 million people globally have PAOD and the number is continuously increasing. PAOD often causes tissue necrosis and may require the need for limb amputation. Approximately 150,000 Americans undergo leg or foot amputations annually due to PAOD complications. The diagnosis of PAOD is based on history, physical examination, and measurement of ankle brachial index. Segmental limb pressures and duplex ultrasound studies can help to define the extent of PAOD and noninvasive imaging with CT or MR angiography can identify the location and severity of blockages to help identify candidates for revascularization. Invasive angiography however remains the gold standard for diagnosis because angiography has the best resolution for anatomic and hemodynamic assessment of PAOD obstructions.

A large percentage of PAOD patients suffer from chronic total occlusions (CTOs) where the artery lumen is completely blocked. In these CTO cases, insufficient blood flow results in tissue necrosis, chronic non-healing ulcers, and risk of infection and amputation. To treat CTO stenosis with endovascular methods, the angioplasty guide wire must pass through the CTO stenosis connecting the upstream arterial lumen with the downstream arterial lumen. With advances in physician experience and technology, including specialized catheters, wires, and reentry devices, many PAOD blockages can be treated using endovascular methods.

Although percutaneous therapies for PAOD have evolved, chronic total occlusions (CTOs) (complete blockages) comprised of dense fibrotic and calcified tissue are difficult to treat with traditional percutaneous transluminal angioplasty (PTA) techniques and tools. In many cases, the impenetrable proximal cap of the calcified CTO prevents guidewire passage and results in wire deflection into the wall of the artery (subintimal) or into the extravascular space (perforation), causing additional complications.

Laser, Shockwave Intravascular Lithotripsy, and Mechanical atherectomy methods (i.e., rotablator) are effective for some CTOs, but these ablative devices carry significant risk of arterial dissection and/or perforation, and end with no reflow. Because of the limitations of percutaneous or abrasive revascularization, many CTO patients require open surgical bypass procedures which have significant morbidity and require extensive recovery time.

U.S. Pat. Nos. 10,342,902; 11,890,259; and 2024/0090960 are incorporated herein by this reference.

### BRIEF SUMMARY OF THE INVENTION

Featured is a system and method which drives an impacting member (e.g., wire) designed to weaken the structural integrity of an occlusion in an artery thereafter enabling safe guide wire passage. The operator positions the impacting member wire to the location of the CTO through a catheter under fluoroscopic imaging guidance. One potential problem, though, is when the impacting wire is deployed far into the artery and oscillated back and forth, the operator may not know if the wire is striking plaque in the artery or, instead, the arterial wall causing potential damage to the artery. In one embodiment, that problem is addressed by measuring the force imparted by the wire. A low impact force would indicate that the wire is striking the artery wall. In response, a number of actions can be taken such as alerting the operator and/or stopping the mechanical action of the wire. Also, when the integrity of the plaque is compromised (cracking or a small hole drilled into it), the impact force imparted by the wire will drop also allowing a number of different actions to be carried out. Therefore, this novel device improves CTO luminal crossing, to reduce procedural complexity, and to improve short and long-term patient outcomes.

The problem of penetrating the chronic total occlusions (CTOs) in arteries without damaging normal elastic arterial tissue and thus thereafter enabling safe guide-wire passage is solved using a novel probe based on a computer controlled electro-mechanical impactor (EMI) that assesses tissue compliance, self-adjusts the impact force, and breaks apart the arterial occlusion. The EMI provides a series of rapid, repeated, direct impact pressure events on the CTO using a “jackhammer” to modify the CTO plaque without damaging normal elastic arterial tissue and thus to enable safe guide-wire passage. Guidewire passage allows the introduction of arterial blockage treatment devices such as balloon angioplasty. Without CTO plaque modification a guidewire cannot be inserted into the blockage to enable treatment.

Reduction to practice has been demonstrated. A benchtop instrument was fabricated, and the technology was demonstrated on phantoms simulating CTOs, on human artery segments with CTOs, and on human cadaver legs with CTOs. Ten femoral human artery segments, approximately 3 to 5 inches in length, were purchased from Science Care, 2001 W. Pinnacle Peak Rd., Ste. 175, Phoenix, AZ 85027. These specimens were obtained by Science Care from tissue repository banks located in various hospitals across the US. The provided specimens were screened for guidewire passage. Eight of them had CTOs while two had partial occlusions. The artery segments were placed into a fixture to hold them in place. The fixture consisted of two Plexiglas plates with practiced channels, into which the artery segments were placed and slightly compressed. A small piece of gauze was placed underneath the artery to secure it in place and then the impacting wire was placed against the CTO. The amplitude and the frequency of the impact pulses were continuously monitored. The frequency of the impact pulses and the magnitude of the impact force were significantly lower in the areas of the artery with mild partial occlusions than in the areas with total occlusions. The average impact force in the non-occluded areas was 0.6N+/−0.28N, while in the calcified areas the impact force was 1.4N+/−0.24N. Wire passage was successfully enabled using the impactor in 7 out of the 8 arteries with CTOs (>87% success rate). One artery had a CTO with a length of over 25 mm, which was not penetrated following impactor application. Experiments were performed on cadaver amputated legs with CTOs. Out of the 5 screened legs, only one showed calcifications in the CT fluoroscopy image. The catheter with the impact wire was placed against CTO under fluoroscopy guidance. The passage of the impacting wire was rapidly performed after <100 impacts. It appeared that the occlusion was only partial. Post procedural dissection showed minimal calcifications and a soft CTO.

The performed experiments indicate that the EMI technique creates a directed impact pressure to modify the CTO plaque without damaging the normal elastic arterial tissue enabling safe guide-wire passage. This is possible by using a tissue compliance assessment device that reduces the impact force in the case of the presence of soft tissue. Therefore, the EMI device provides favorable efficacy and safety compared to available CTO treatment devices. The EMI will be applicable to both remote arterial blockages (e.g., legs) as well as cardiac blockages.

Featured is an occlusion penetrating system comprising a probe including an impact member, an actuator for vibrating the impact member, and a sensor configured to measure the impact force of the impact member. A catheter delivers the impact member within a tube (e.g., an artery). A power supply is connected to the actuator for energizing the actuator and a processing subsystem is responsive to the measured impact force of the impact member and configured to ascertain whether the impact member is impacting the tube or an occlusion (e.g., plaque) in the tube and to automatically deenergize the actuator when the measured impact force indicates the impact member is impacting the tube.

In one version, the sensor is a load cell associated with the actuator. Alternatively or in addition, the sensor is associated with the impact member, for example a Bragg sensor inside a hollow impact member.

The probe actuator can be a solenoid with an electromagnetic armature and the impact member is a wire mechanically connected to the solenoid armature. Preferably, the power supply is controllable to adjust the frequency and duty cycle of the actuator.

Preferably, a measured impact force above a predetermined threshold is processed by the processing subsystem to indicate the impact member is striking plaque in the artery and a measured output force below a predetermined threshold is processed by the processing subsystem to indicate the impact member is striking the artery. The processing subsystem can also be configured to notify an operator when a measured impact force indicates the impact member is striking the tube, striking the occlusion in the tube, and/or striking an occlusion which has reached structural failure. In some embodiments, the processing subsystem is further configured to deenergize the power supply when a measured impact force indicates the impact member is striking the tube and/or striking an occlusion which has reached structural failure.

In one version, the impact member is hollow and includes an imaging catheter therein. The imaging catheter is preferably rotatable relative to the impact member and the processing subsystem can be further configured to process image signals transmitted by the imaging catheter.

In some embodiments, the exposure of the impact member relative to the catheter is adjustable and the outer catheter includes a balloon thereabout for centering the catheter in the tube.

Also featured is an occlusion penetrating method comprising inserting a catheter about an impact member into a tube (e.g., vasculature), driving the impact member out of the catheter, vibrating the impact member, measuring an impact force of the impact member, and determining whether the impact member is impacting the tube or an occlusion in the tube. If the impact member is impacting the tube the impact member is prevented from impacting the tube and if the impact member is impacting the occlusion, the impact member is driven until the occlusion reaches structural failure.

The method may further include performing endovascular stenosis treatment on the structurally failed occlusion. Driving the impact member may include adjusting the frequency and duty cycle of the vibrations.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram an exemplary CTO management apparatus;

FIG. 2A-2B depict one design of an EMI probe;

FIGS. 3A-3D depict another version of an EMI probe;

FIG. 4A shows an impact member striking a tube;

FIG. 4B shows an impact member striking an occlusion in the tube;

FIG. 5 is a flow chart showing exemplary methods and process algorithms associated with the disclosure;

FIG. 6 shows an EMI device including the controller and the probe;

FIG. 7 are photographs of the CTO phantoms testing apparatus;

FIG. 8 shows an example of a human artery segment with a CTO;

FIG. 9 shows results of the human artery impacting experiment; and

FIGS. 10A-10B are a photograph of the catheter delivery through the amputated leg and a fluoroscopy image showing wire passage through a CTO.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

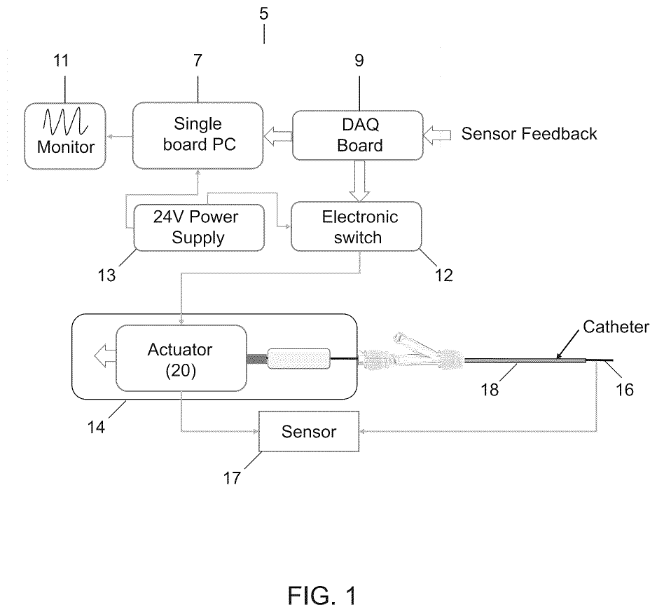

An occlusion penetrating system includes the probe 14, FIG. 1 with an impact member such as nitinol flexible wire or member 16 and an actuator 20 such as a solenoid for actuating the impact member. A sensor 17 such as a recoil sensor is placed either at the electromagnet moving shaft and/or further down within the impact wire (in which case the sensor can be a fiber Bragg or other type of force sensor). The sensor is configured to measure the impact force of the impact member when deployed in a tube such as vasculature. Catheter 18 delivers the impact member within a tube such as an artery.

Power supply 13 is connected to EMI probe 14 via an electronic switch 12 for energizing the actuator 20. Processing subsystem 5 which can include a computer or a specialized microcontroller) 7 is responsive to the measured impact force output from sensor 17 (e.g., via data acquisition board 9) of the impact member and configured to ascertain whether the impact member is impacting the tube or an occlusion in the tube and to automatically deenergize the actuator when the measured impact force indicates the impact member is impacting the tube. Monitor 11 may be used to display the impact force of the impact member.

Sensor 17 can be a load cell associated with the actuator or a strain sensor associated with the impact member (e.g., a Bragg sensor) (e.g., attached between the impact wire and the probe).

A controller unit including a computer-controlled voltage pulse generator (electronic switch circuit) 12 with adjustable frequency (1-40 Hz) and adjustable duty cycle (10-50%) sends pulses to EMI probe 14 actuator 20. The probe can be based on a solenoid that actuates a wire 16 that is passed through lumen of a catheter 18 which is placed in the artery against the CTO.

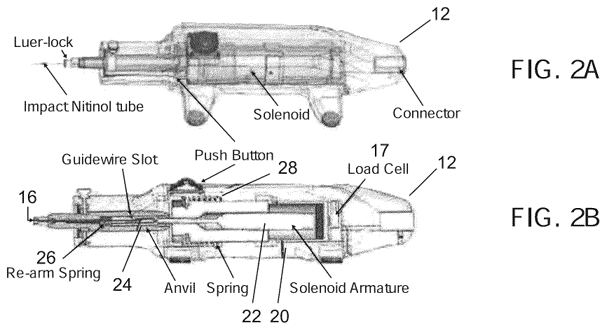

When an electric impulse is applied to the solenoid coil 20, FIGS. 2A-2B, its armature 22 is actuated and moves the shaft 23. The impact wire 16 is attached (mechanically connected) to the shaft and thus it is pushed forward with a force proportional to the current applied to the solenoid coil. Thus, the impact force of the solenoid is transmitted to the impact wire to penetrate the CTO after a series of impacts. A spring 26 is placed at the distal end of the anvil to push back the solenoid armature and the impact wire after each impact and prepare them for a new impact. In this example, a load cell 17 is placed at the proximal end of the solenoid armature to measure the recoil force. The distal end of the solenoid armature is placed against a low force spring 28 which maintains positive contact between the solenoid and load cell during actuation.

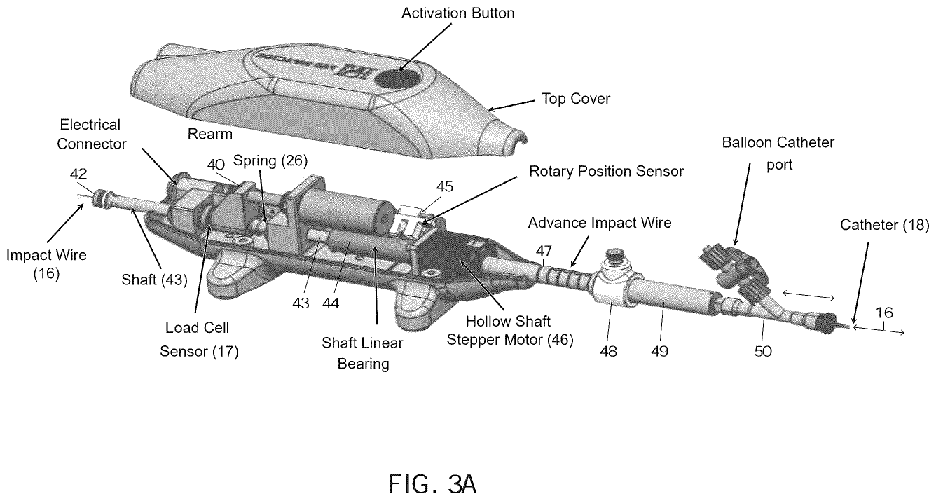

In another embodiment shown in FIGS. 3A-3D, probe 12′ includes an actuator (e.g., a solenoid with armature 22 connected to block 40) which vibrates block 40 back and forth. Impact wire 16 is releasably locked (e.g., via a luer-lock 42) inside shaft 43 which is coupled inside block 40 so now both wire 16 and shaft 43 vibrate. Thus, in this version, the impact wire is easy to replace if its tip gets damaged.

Shaft 43 and impact wire 16 proceed through linear bearing 44 and stepper motor 46 which functions to rotate the shaft and impact wire. This functionality enables the acquisition of ultrasound (US) or optical coherence to tomography (OCT) images, when such imaging catheters are embedded within the lumen of a hollow impact wire. The combined axial and angular movements enable the collection of 3D images of the plaque. Rotary position sensor 45 functions to indicate the homing position of the shaft and impact wire, such that the rotation can be started every time from the same angular position. Another position sensor (optical linear encoder) can be used to monitor the axial movement of the shaft, which can have attached an optical scale to be read by the encoder. The signals from this encoder can be used to trigger OCT data acquisition and generate OCT images.

Adjustor 48 adjusts the extent of impact wire exiting the outer catheter 18 distal end (see FIGS. 3B-3C) as outer shaft 49 is slidable about inner shaft 47 which is disposed about the impact wire. Thus, adjuster 48 is releasably coupled to shaft 49 in different positions relative to shaft 47.

Impact wire 16 passes through lumen 50 and outer catheter 18 which may include balloon 50 to center the catheter in the artery and to direct the impact wire 16 to the middle of a CTO.

As shown in FIG. 3D, impact wire 16, in this particular version, is hollow and includes OCT imaging catheter 60 rotatable therein and terminating in ball lens 62 for imaging an artery and a CTO through opening 64 in impact wire 16.

Sensor 17 is now inside impact wire 16 and functions to measure the force imparted on wire 17 and to send signals representing the same through the OCT catheter to processing subsystem 5, FIG. 1.

As the Bragg sensor 17 generates an optical wavelength shift when is compressed, its ends are epoxied to the impact wire, while the impact wire in this section is helically cut as shown at 67 to enable sensor compression between epoxy anchors 66 and 68.

Also, the EMI impactor stepper motor 46 allows the impact wire to pass by while also enabling shaft rotation. This functionality enables the use of the OCT catheter that can be rotated to explore plaque morphology changes after each impact. The use of the fiber OCT catheter also enables the insertion of a fiber Bragg sensor 17.

The EMI unit may also be equipped with an impact wire exposure adjustment mechanism 48 which uses a sliding tube to move the catheter forth and back and exposure more or less length of the impacting wire, depending on the length of the CTO. The locked position can then be secured with a screw-based system.

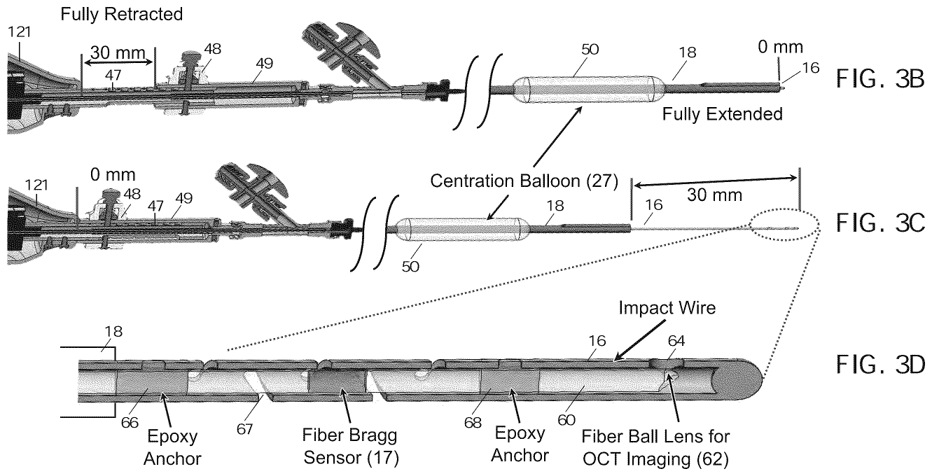

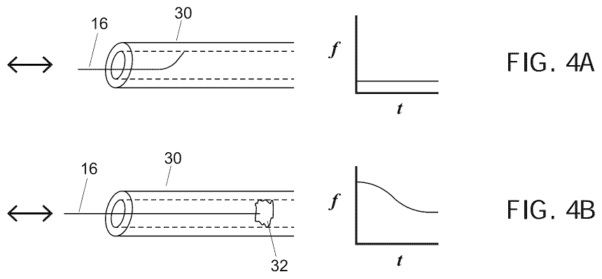

The impact force of the wire on the substrate depends on the compliance of the tissue. For soft tissue, the movement of the impact wire is dampened by movement of the tissue (e.g., artery 30, FIG. 4A) and thus the force detected by the load cell is minimal. When the wire impacts hard surface, such as CTO 32, FIG. 4B, the impact wire comes to an abrupt halt resulting in a high impact force. The impact force can be continuously displayed on monitor 11, FIG. 1. Thus, the system is configured to immediately stop impacting the artery when the force signal is low and reposition the catheter to avoid arterial wall perforation or damage.

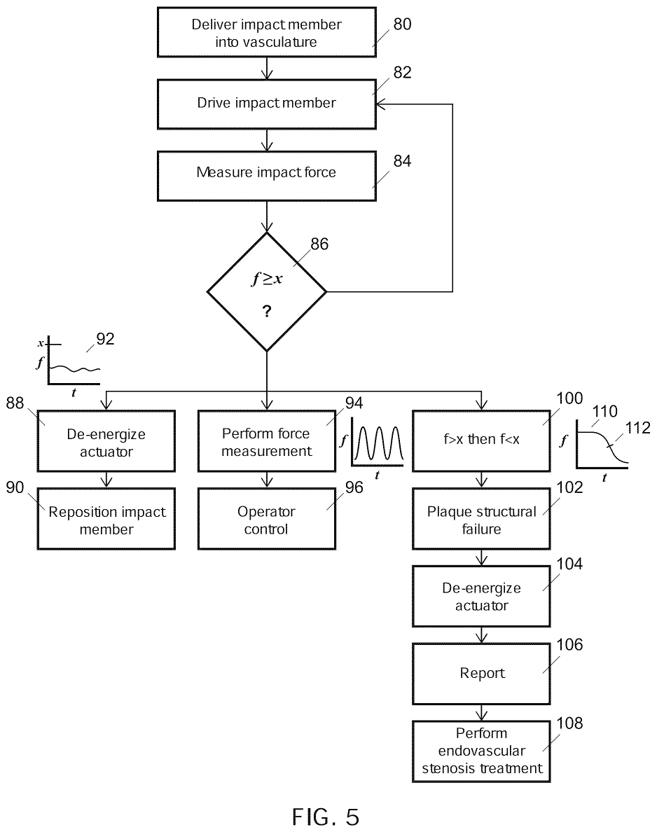

Thus, the impact member is delivered into vasculature, step 80, FIG. 5 and rapidly driven axially back and forth, step 82. The impact force of the impact member is measured, step 84 and when the impact force is less than a preset value (e.g., 0.5N), step 86, the system (e.g., software algorithms operating on PC 7, FIG. 1) (or other computer types, processors, controllers, application specific integrated circuitry, and the like) can automatically take a number of actions such as deenergizing the actuator, step 88 (by controlling switch 12, FIG. 1) so the operator can reposition the impact member, step 90 in clear cases as shown at 92 where the impact member is striking a vessel wall and the measured impact force is very low. Another choice is to plot the force measurements, step 94 (on monitor 11, FIG. 1) in order for the operator to control, for example, the position of the impact member, the pulse frequency, amplitude, and the like, step 96, and then make decisions based on the impact force measurements.

Step 100-106 and graph 110 show an impact member striking plaque (f>x) (e.g., above 1.4N) and then the force lowers to below x after indicating plaque structural failure, step 102 (e.g., a fracture of the plaque or a small hole drilled through it as shown at 112) whereupon the system automatically deenergizes the actuator, step 104, and reports the success of the procedure, step 106.

The plaque is preferably not broken up and only microscopic plaque particles are created. Instead, the structural integrity of the plaque is compromised (softened and cracks are being created) by the new system so a guide wire of an endovascular stenosis treatment apparatus can now pass through the plaque, step 108.

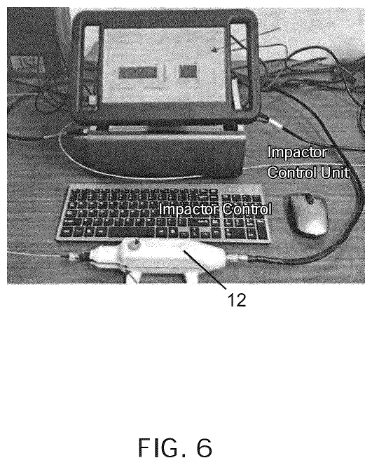

One reduction to practice of the EMI device is shown in FIG. 6. The EMI device may use an electromechanical actuated wire residing within a 5 French cannula (1.6 mm diameter), which is coupled to the probe. A control unit produces a controlled burst of 1-40 Hz voltage pulses with an adjustable duty cycle (10-50%) which are applied to a solenoid residing within the probe. The duty cycle adjustment enables the adjustment of the impact force.

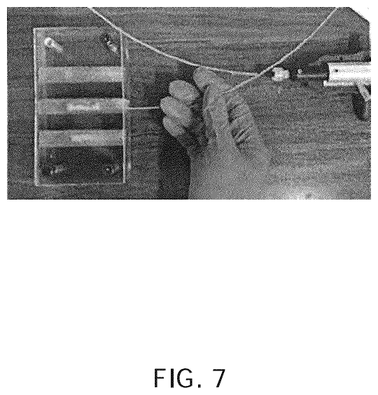

Device testing on phantoms is shown in FIG. 7. Pieces of bovine arteries were filled up with a mixture of silicone gel and dental cement to create occlusions with similar mechanical properties of a human CTO, with compressive strength >0.8 MPa. Since the hardness of the plaque varies from one sample to the next, there is limited data regarding the precise mechanical properties that covers all samples. However, experimental data in literature indicates the puncture force required to penetrate the plaque ranges from 0.08 to 2.5 N. To stabilize the sample, a small piece of gauze was placed underneath the artery to fill up any void space and secure it in place. The operator of the device turned the device to the working mode and started to impact the artery lumen by pressing the button of the probe while the second operator was constantly pushing the catheter with the actuated wire towards the other end of the artery segment.

For each impact, a wire velocity of >800 mm/sec was obtained with an impact pressure of 5-15 MPa. Repeated impacts (up to 100 impacts with peak force <1 N and up to 250 impacts with a peak force ranging from 1 to 2 N) enabled the fracture of 5 mm thick occlusions and thus guidewire passage. During this experiment, 9 specimens with different compressive strength values were investigated. A summary of the experiments is shown in Table 1.

TABLE 1
Phantom Testing Summary

| | | | Number of |
|---|---|---|---|
| | | Measured | pulses that |
| | Compressive | impact | enabled impact |
| | Strength [MPa] | force [N] | wire passage |
| Specimen Class 1 | 1.22 +/− 0.24 | 1.2 +/− 0.2 | 85 +/− 20 |
| Specimen Class 2 | 3.45 +/− 0.42 | 1.5 +/− 0.3 | 120 +/− 45 |
| Specimen Class 2 | 6.80 +/− 0.37 | 1.8 +/− 0.3 | 220 +/− 24 |

A pilot study in segments of human arteries with CTOs as well as in amputated legs was also performed to demonstrate the viability of this technology for managing CTOs.

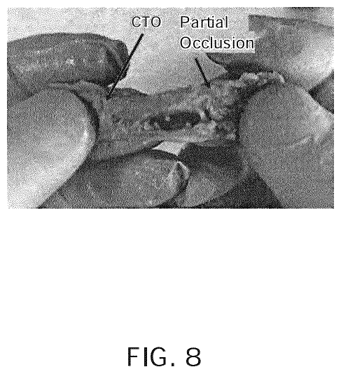

Ten femoral human artery segments, approximately 3 to 5 inches in length, were purchased from Science Care, 2001 W. Pinnacle Peak Rd., Ste. 175, Phoenix, AZ 85027. These specimens were obtained by Science Care from tissue repository banks located in various hospitals across the US. The specimens were screened for guidewire passage. Eight of them had CTOs while two had partial occlusions. An example of a sectioned arterial segment with a CTO and a partial occlusion is shown in FIG. 8.

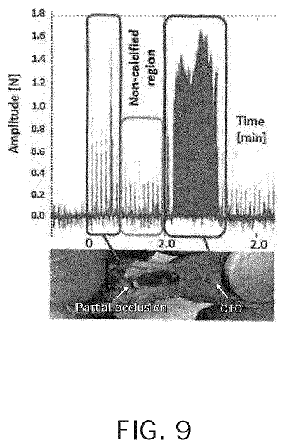

The artery segments were placed into a fixture to hold them in place. The fixture included two Plexiglas plates with practiced channels, into which the artery segments were placed and slightly compressed. Similar to the phantom testing procedure, a small piece of gauze was placed underneath the artery to secure it in place, and then the impacting wire was placed against the CTO. The amplitude and the frequency of the impact pulses were continuously monitored. As measured and shown in FIG. 9, the frequency of the impact pulses and the magnitude of the impact force were significantly lower in the areas of the artery with mild partial occlusions compared to the areas with total occlusions. The average impact force in the non-occluded areas was 0.6N+/−0.28N, while in the calcified areas the applied force was 1.4N+/−0.24N.

Wire passage was successful in 7 out of the 8 arteries with CTOs (>87% success rate). One artery had a CTO with a length of greater than 25 mm, which was not penetrated using the EMI device.

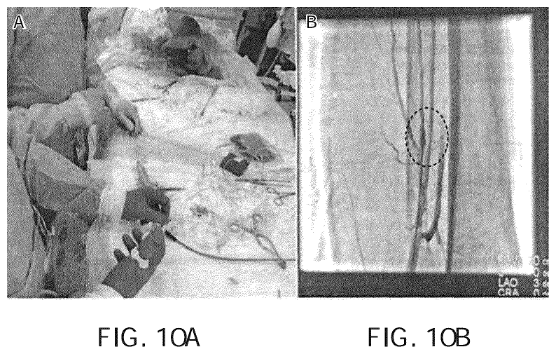

Experiments were performed to demonstrate guidewire passage in amputated legs with CTOs. These experiments were performed at Brigham and Women's Hospital, Boston. Out of the 5 screened legs, only one showed calcifications in the CT fluoroscopy image (see FIGS. 10A-10B). The catheter with the impact wire was placed against CTO under fluoroscopy guidance. The passage of the impacting wire was rapidly performed, after <100 impacts. It appeared that the occlusion was only partial based on postprocedural dissection which showed minimal calcifications.

One innovation of the subject technology is its characteristic enhanced, preferential action on hard (brittle, inelastic) vs. soft (compliant/elastic) tissues. The impact force is continuously monitored to prevent tissue damage. Additionally, the impact force is software monitored. The software can be configured to instantly reduce the frequency and duty cycle of the pulses sent to the probe when the impact force of 3-5 consecutive pulses is below a user selectable threshold. Thus, the EMI modifies hard, noncompliant CTO plaques, but will not damage normal complaint arterial tissue. The EMI preferential treatment effect on calcific tissue, with no damage to normal tissue, offers a desirable safety margin against artery wall damage, subintimal entry, and perforation when penetrating calcific CTOs.

In summary, the new approach and EMI device enables: (1) more efficient blockage penetration and lesion crossing; (2) higher success rates of guide-wire placement; and (3) overall improved probe efficacy and safety of the CTO management procedure.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. An occlusion penetrating system comprising:
a probe including:
an impact member,
an actuator for vibrating the impact member, and
a sensor configured to measure the impact force of the impact member;
a catheter for delivering the impact member within a tube;
a power supply connected to the actuator for energizing the actuator; and
a processing subsystem responsive to the measured impact force of the impact member and configured to ascertain whether the impact member is impacting the tube or an occlusion in the tube and to automatically deenergize the actuator when the measured impact force indicates the impact member is impacting the tube.

2. The system of claim 1 in which the sensor is a load cell associated with the actuator.

3. The system of claim 1 in which the sensor is associated with the impact member.

4. The system of claim 3 in which the sensor inside the impact member.

5. The system of claim 1 in which the probe actuator is a solenoid with an electromagnetic armature and the impact member is a wire mechanically connected to the solenoid armature.

6. The system of claim 1 in which the power supply is controllable to adjust the frequency and duty cycle of the actuator.

7. The system of claim 1 in which the tube is an artery and the occlusion is plaque in the artery.

8. The system of claim 7 in which a measured impact force above a predetermined threshold is processed by the processing subsystem to indicate the impact member is striking plaque in the artery and a measured output force below a predetermined threshold is processed by the processing subsystem to indicate the impact member is striking the artery.

9. The system of claim 1 in which the processing subsystem is configured to notify an operator when a measured impact force indicates the impact member is striking the tube, striking the occlusion in the tube, and/or striking an occlusion which has reached structural failure.

10. The system of claim 9 in which the processing subsystem is further configured to deenergize the power supply when a measured impact force indicates the impact member is striking the tube and/or striking an occlusion which has reached structural failure.

11. The system of claim 1 in which the impact member is hollow and includes an imaging catheter therein.

12. The system of claim 11 in which the imaging catheter is rotatable relative to the impact member.

13. The system of claim 11 in which the processing subsystem is further configured to process image signals transmitted by the imaging catheter.

14. The system of claim 1 in which an exposure of the impact member relative to the catheter is adjustable.

15. The system of claim 1 in which the catheter includes a balloon thereabout for centering the catheter in the tube.

16. An occlusion penetrating method comprising:
inserting a catheter about an impact member into a tube;
driving the impact member out of the catheter;
vibrating the impact member;
measuring an impact force of the impact member;
determining whether the impact member is impacting the tube or an occlusion in the tube;
if the impact member is impacting the tube, stopping the impact member from impacting the tube; and
if the impact member is impacting the occlusion, vibrating the impact member until the occlusion reaches structural failure.

17. The method of claim 16 in which the tube is vasculature.

18. The method of claim 16 further including performing endovascular stenosis treatment on the structurally failed occlusion.

19. The method of claim 16 in which vibrating the impact member includes adjusting the frequency and duty cycle of the vibrations.

20. The method of claim 16 in which a measured impact force above a predetermined threshold indicates the impact member is striking an occlusion and a measured output force below the predetermined threshold indicates the impact member is striking the tube.

21. The method of claim 16 further including notifying an operator when a measured impact force indicates the impact member is striking the tube, striking the occlusion in the tube, and/or striking an occlusion which has reached structural failure.

22. The system of claim 16 in which the impact member is hollow and includes an imaging catheter therein.

23. The system of claim 22 in which the imaging catheter is rotatable relative to the impact member.

24. The system of claim 22 in which the processing subsystem is further configured to process image signals transmitted by the imaging catheter.

25. The system of claim 16 in which an exposure of the impact member relative to the catheter is adjustable.

26. The system of claim 16 in which the catheter includes a balloon thereabout for centering the catheter in the tube.
